(19) United States
(12) Patent Application Publication
(10) Pub. No.: US 2012/0245491 A1
(43) Pub. Date: Sep. 27, 2012

(54) POSTURE FEEDBACK SYSTEM AND METHOD, DETACHABLE TRACTION SYSTEM, AND TRACTION SAFETY BELT COMBINATION

(75) Inventors: FREDRIK AMELL, Huntington, NY (US); Elan Blum, New York, NY (US)

(73) Assignee: Novel Ergonomics LLC, New York, NY (US)

(21) Appl. No.: 13/294,433

(22) Filed: Nov. 11, 2011

Related U.S. Application Data
(60) Provisional application No. 61/465,824, filed on Mar. 25, 2011.

(51) Int. Cl. A61B 5/103

(52) U.S. Cl. 600/595

(57) ABSTRACT

A device for monitoring a posture of a user is provided. The device includes a belt adapted to encircle a torso of the user and provide an upward force to a spinal region of the user in response to a signal from a controller, and at least one sensor adapted to provide at least one measurement signal to the controller. The device also includes a feedback system activated by the controller to alert the user that a posture of the user is determined to be outside an acceptable range based on the at least one measurement signal. An ergonomic posture assisting system is provided that includes a detachable belt. A detachable belt is provided that includes a locking mechanism adapted to prevent the attachment mechanism from disconnecting in response to a signal. A posture feedback system and a device for monitoring a posture of a user are provided.

### CROSS-REFERENCE TO RELATED APPLICATIONS

This application claims the benefit of U.S. Provisional Application No. 61/465,824 filed Mar. 25, 2011, which is incorporated herein by reference.

### STATEMENT REGARDING FEDERALLY SPONSORED RESEARCH OR DEVELOPMENT

Not Applicable

### BACKGROUND OF THE INVENTION

1. Field of the Invention

The present invention relates to a traction belt system and a posture assistance system, and in particular relates to a detachable belt system for providing posture feedback and assistance in a vehicle and a belt or lumbar support providing posture assistance feedback.

2. Description of the Related Art

Sitting for extended periods may be difficult for some, in particular those with back pain. Poor posture in particular, is known to aggravate back pain, stretch or damage ligaments and may ultimately lead to back pain. Driving long distances, for instance in a long-haul truck requires sitting for long periods. Ergonomic seats have been developed to assist people in sitting, including for instance adjustable and inflatable lumbar supports. Some truck seats may have pneumatic shock absorption systems, along with conventional seat springs. Safety belts typically traverse the hip area of a driver, namely lap belts, and may additionally have a belt crossing over the chest, namely a shoulder belt.

A significant number of drivers and other people who sit for an extended period of time suffer from sitting-related lower back pain. The problem with many posture-correcting devices is their over-reliance on the strength of the user's support muscles to hold the torso erect. Fatigue has been listed among the main reasons for why people lose proper posture and often slouch. Merely sitting properly and thus holding the torso erect, without assistance, for prolonged periods of time may over-exercise support muscles and lead to a cramping that may worsen existing muscle spasms, which is especially true for overweight individuals with heavier torsos. Furthermore, individuals who suffer from sitting-related lower back pain may be incapable of sitting properly without assistance for prolonged periods of time without fatiguing and subsequently slouching.

U.S. Pat. No. 7,097,628 relates to a traction chair system for relieving an individual of lower back pain. The traction chair system includes a chair frame, a seat, a plurality of actuators attached between the chair frame and the seat for elevating/lowering the seat, a backrest, a harness attached to the backrest for securing an individual to the backrest, a motor mechanically elevating/lowering the backrest, and a control unit in communication with the actuators and the motor. A scale is positioned within the seat and in communication with the control unit for providing feedback to the individual regarding the actual amount of traction occurring. The amount of traction being applied to an individual is shown on a display of the control unit.

U.S. Pat. No. 6,827,694 relates to a posture training device for back muscles used to direct training to maintain good posture. A rigid spinal curvature model with sensors and single attachment point is provided with shoulder-based and alternative belt-based embodiments.

U.S. Pat. No. 4,981,131 relates to a passive motion back support that uses continuous passive motion to cycle the position of the user's back. A lumbar support cycles between positions of inflation and deflation to move the spine of a person between degrees of lordosis.

### BRIEF SUMMARY OF THE INVENTION

A device for monitoring a posture of a user is provided. The device includes a belt adapted to encircle a torso of the user and provide an upward force to at least part of a spinal region of the user in response to a signal from a controller, and at least one sensor adapted to provide at least one measurement signal to the controller. The device also includes a feedback system activated by the controller to alert the user that a posture of the user is determined to be outside an acceptable range based on the at least one measurement signal.

The feedback system may signal to the user with at least one of a sound and a vibration. The posture of the user may be determined to be outside the acceptable range when the at least one measurement signal exceeds a first predetermined threshold; when the at least one measurement signal falls below a second predetermined threshold; and/or when the at least one measurement signal is a plurality of measurement signals satisfying a preprogrammed model. After the posture of the user is determined to be outside the acceptable range, at least one of the first predetermined threshold and the second predetermined threshold may be changed based on an adjusted posture of the user. The adjusted posture of the user may be identified based in part on a speed at which the users assumes the adjusted posture after the user is alerted by the feedback system.

The controller may be adapted to signal the belt to provide the upward force to the user when the controller determines that the posture of the user is outside the acceptable range. The belt may be attachable to, and detachable from, a seat back. The belt may provide the upward force by moving an attachment mechanism that couples the belt and at least one of a seat back surface and a lumbar support upward with respect to the seat back surface.

The belt may include: a seat-back apparatus including a motor adapted to provide the upward force; a seat-back apparatus including a pneumatic system adapted to provide the upward force; a seat-back apparatus including a hydraulic system adapted to provide the upward force; a seat-back apparatus including a spring system adapted to provide the upward force; and/or a seat-back apparatus including a rack and pinion system adapted to provide the upward force.

The belt may include a shirt, a vest and/or a jacket, and the at least one sensor may be a plurality of sensors, and at least one of the sensors may be on the shirt, the vest or the jacket.

The at least one sensor may include a pressure sensor and/or a flex sensor.

An ergonomic posture assisting system is provided that includes a belt adapted to encircle a torso of a user and adapted to selectively attach to, and detach from, an attachment mechanism of a seat back surface. The ergonomic posture assisting system also includes at least one sensor adapted to provide at least one measurement signal to a controller, and a posture assistance system controlled by the controller and adapted to provide an upward force to a portion of the belt.

The controller may be adapted to signal to the user when a posture of the user is determined to be outside an acceptable range based on the at least one pressure measurement signal. The controller may signal to the user when a posture of the user is determined to be outside the acceptable range using at least one of a sound and a vibration. The posture assistance system may provide the upward force to the portion of the belt by moving the attachment mechanism upward with respect to the back surface of the seat. The system may further include the controller, and the controller may communicate with the posture assistance system.

The attachment mechanism of the seat back surface may be coupled to at least one vertically oriented rail in the seat. The attachment mechanism may be adapted to move up and down on the at least one vertically oriented rail in response to a motor, a pneumatic system, a hydraulic system, and/or a spring system.

The attachment mechanism of the seat back surface and/or the belt may include a funneling system adapted to guide the belt into engagement with the attachment mechanism. The attachment mechanism of the seat back surface and/or the belt may include a magnet system adapted to facilitate coupling of the attachment mechanism and the belt.

The attachment mechanism may include an electrical connection adapted to transmit power and/or data, and/or an air pressure connection.

The attachment mechanism may include a locking mechanism adapted to respond to a collision sensor, a speed sensor, and/or an accelerometer.

A method for assisting posture of a user is provided that includes selecting an assistance amount, and providing an upward force to a torso of the user. An amount of the upward force is based on the selected assistance amount and varies over time.

The method may include determining a measurement signal corresponding to a pressure between the user and a seat cushion and/or a seat back surface when the user is prompted. The amount of the upward force may be further based on the measurement signal.

The measurement signal may be determined by a pressure sensor and/or a flex sensor. The measurement signal may be a plurality of measurement signals.

The method may include providing a feedback signal to the user based on the measurement signal. The feedback signal may include a sound and/or a vibration.

The feedback signal may be provided to alert the user that a posture of the user is determined to be outside an acceptable range. After the posture of the user is determined to be outside the acceptable range, the method may further include determining a further measurement signal corresponding to a further pressure between the user and the seat when the user sits in the seat in an adjusted posture.

The adjusted posture of the user may be identified based in part on a speed at which the users assumes the adjusted posture after the feedback signal is provided to the user.

The assistance amount may be selected by the user and/or a controller having a memory from a prior use by the user.

A device is provided that includes a belt adapted to encircle a torso of a user, and an attachment mechanism adapted to removably attach the belt to a vehicle. The device also includes a locking mechanism adapted to prevent the attachment mechanism from disconnecting in response to a signal.

The locking mechanism may be adapted to prevent the attachment mechanism from disconnecting in response to a signal from a sensor in the vehicle. The sensor may include a collision sensor, an accelerometer, and/or a vehicle speed sensor. The signal from the sensor may be in response to a collision force, an acceleration, a deceleration, and/or a vehicle speed.

The belt may include a size-adjustable internal belt along a bottom portion that is adapted to distribute force over the user when the belt is attached to the vehicle.

At least a portion of the attachment mechanism may be situated on a back surface of the belt. At least a portion of the attachment mechanism may include a seat including a bottom surface and a back surface. The seat may be attached to the vehicle.

The device may be adapted to aid a posture of the user, and the device may be further adapted to provide an upward force to at least part of a spinal region of the user in response to a signal from a controller. The device may further include at least one pressure sensor adapted to provide at least one pressure measurement signal to the controller. The belt may include a shirt, a vest, and/or a jacket.

A posture feedback system is provided that includes a lumbar support adapted to be positioned on a chair, and a pressure sensor arranged on the lumbar support. The posture feedback system also includes a controller adapted to receive a measurement signal from a touch detecting sensor, and a feedback arrangement controlled by the controller.

The touch detecting sensor may include a flex sensor and/or a pressure sensor. The touch-detecting sensor may include a plurality of flex sensors and/or a plurality of pressure sensors. The lumbar support may include a chair back.

A deviation in posture of a user above a preset threshold may activate the feedback arrangement. The feedback arrangement may be a sound source and/or a vibration source.

A device for monitoring a posture of a user is provided that includes a belt adapted to encircle a torso of the user, and at least one flex sensor adapted to provide a measurement signal to the controller. The device further includes a feedback system activated by the controller to alert the user that a posture of the user is determined to be outside an acceptable range based on the measurement signal. The feedback system may signals to the user with a sound and/or a vibration.

The posture of the user may be determined to be outside the acceptable range when: the measurement signal exceeds a first predetermined threshold; the measurement signal falls below a second predetermined threshold; and/or the measurement signal is a plurality of measurement signals satisfying a preprogrammed model.

These objects and the details of the invention will be apparent from the following description and accompanying drawings.

### BRIEF DESCRIPTION OF THE DRAWINGS

FIG. 1 is a perspective view of an exemplary embodiment of the present invention for attachment to an existing vehicle seat shown in an attached condition;

FIG. 2 is a perspective view of some of the internal elements of the exemplary embodiment of the present invention shown in FIG. 1;

FIG. 3 is a perspective view of another exemplary embodiment of the present invention integrated with a vehicle seat;

FIG. 4 is a perspective view of some of the internal elements of the exemplary embodiment of the present invention shown in FIG. 3;

FIG. 5 is a front perspective view of another exemplary embodiment of the present invention including a detachable posture feedback belt;

FIG. 6 is a rear perspective view of the exemplary embodiment of the present invention shown in FIG. 5 illustrating the connection mechanism;

FIG. 7 illustrates a method according to an exemplary embodiment;

FIG. 8 illustrates a computer system according to an exemplary embodiment;

FIG. 9 is a side view of a belt and seatback stand attached to an existing vehicle seat; and

FIG. 10 illustrates an office chair according to an exemplary embodiment.

### DETAILED DESCRIPTION OF THE INVENTION

The present disclosure relates to a system designed to help alleviate back pain by unloading weight from the lumbar region of the back while promoting proper posture through feedback. The system also may provide a wake-up mechanism for drowsy drivers. More particularly, the invention may provide pressure on a user's lumbar region, via pressurized air, another gas, or any appropriate fluid, or via a mechanical arrangement, and also generates upward force that reduces the amount of weight and pressure on the user's lower back. The system also encourages proper posture through posture-related tactile feedback to inform the user when their posture has decreased below a preset level. This feedback mechanism serves the dual purpose of posture correction and a wake-up mechanism to alert drivers.

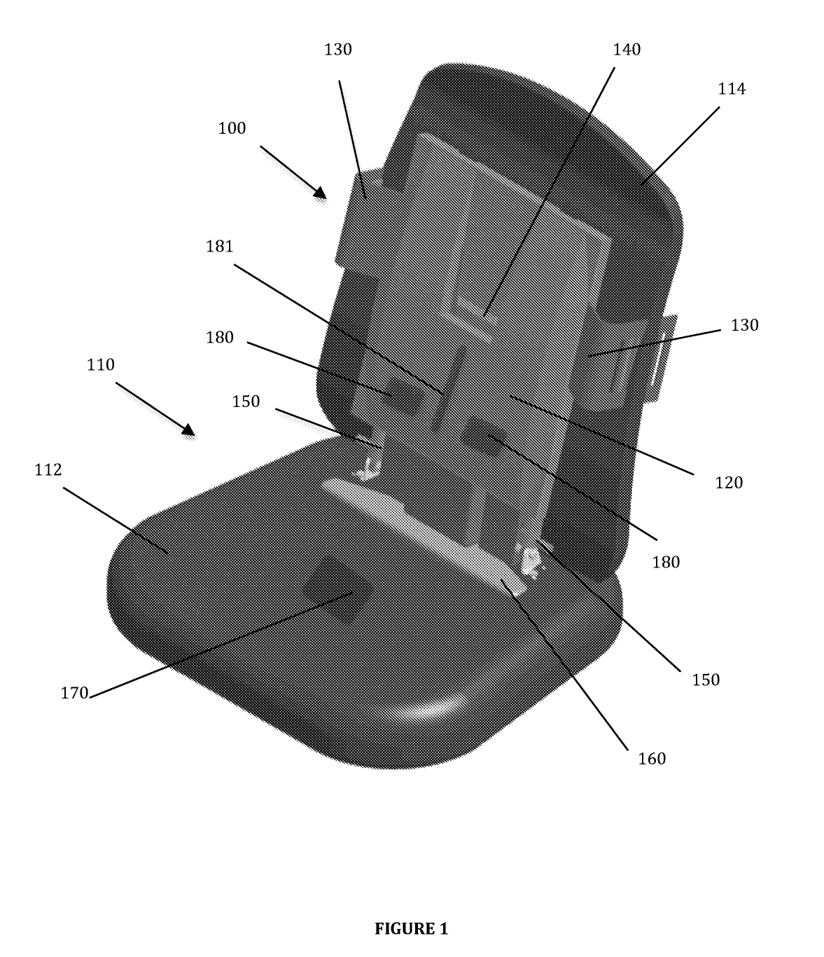

FIG. 1 is a perspective view of an exemplary embodiment of posture assistance stand 100 attached to vehicle seat 110. Alternatively, posture assistance stand 100 may be used with a seat that is not in a vehicle. Posture assistance stand 100 attaches via clips 130 to seat back surface 114 of seat 110. Clips 130 may include a plastic or metal part that may be curved or angled that may extend around a back side of seat back surface 114, and which may adjustably connect to posture assistance stand 100 to enable a tight friction fit between the parts of clips 130 and the back side of seat back surface 114 to be formed, to firmly hold posture assistance stand 100 in place on seat 110. Clips 130 may additionally or alternatively include a strap extending around a back side of seat back surface 114, may include Velcro, may attach to an anchor point on seat back surface 114, or may attach by any other appropriate method. Though two clips 130 are shown in FIG. 1, one clip 130 or more than two clips 130 may also be appropriate. Base 160 of posture assistance stand 100 may rest against seat bottom surface 112 of seat 100 or may wedge between seat back surface 114 and seat bottom surface 112. Base 160 may alternatively attach to seat bottom surface 112 using a strap extending around a bottom side of seat bottom surface 112, may include Velcro, may attach to an anchor point on seat bottom surface 112, or may attach by any other appropriate method. Posture assistance stand 100 may include a lumbar support 120, which may include foam, nylon, neoprene or any other appropriate material. Lumbar support 120 may also be adjustably inflatable. Lumbar support 120 may be designed to rest against a lumbar area of a user when the posture assistance stand 100 is attached to seat 110 and the user sits in seat 110. Lumbar support 120 includes female adapter 140, which alternatively may be a male adapter. Female adapter 140 may include magnets, electromagnets, or any other appropriate locking pieces adapted to cooperate with an adapter to connect to a detachable posture feedback belt 500.

Posture assistance stand 100 also includes pistons 150, which may be air pistons or alternatively may be a motor, springs, an actuator, a rack and pinion system or a combination of two or more of these elements. In particular, a motor and spring combination may be used. Pistons 150 may operate to move lumbar support 120 and female adapter 140 up and down with respect to base 160. Alternatively, pistons 150 may operate to move only female adapter 140 up and down with respect to base 160, and lumbar support 120 may be fixed with respect to base 160. Female adapter 140 may be height adjustable and thus may allow the user to select its starting position. Pistons 150 may move lumbar support 120, including female adapter 140, up and down in response to a signal from a controller that may be in posture assistance stand 100, may be in the vehicle in which seat 110 is located, may be in seat 110, or may be remotely located and connected wirelessly to pistons 150. Pistons 150 may be one or more pistons. The controller operating pistons 150 may receive signals from gluteal pressure sensor 170, which may be on top of or under seat bottom surface 112, and which may be one or more pressure sensors. Additionally, the controller operating pistons 150 may receive signals from back pressure sensors 180 or flex sensor 181, which may be on lumbar support 120, on a detachable support belt, another part of the surface of posture assistance stand 100, or may be under a cushion of seat back surface 114, and which may be one or more pressure sensors or flex sensors.

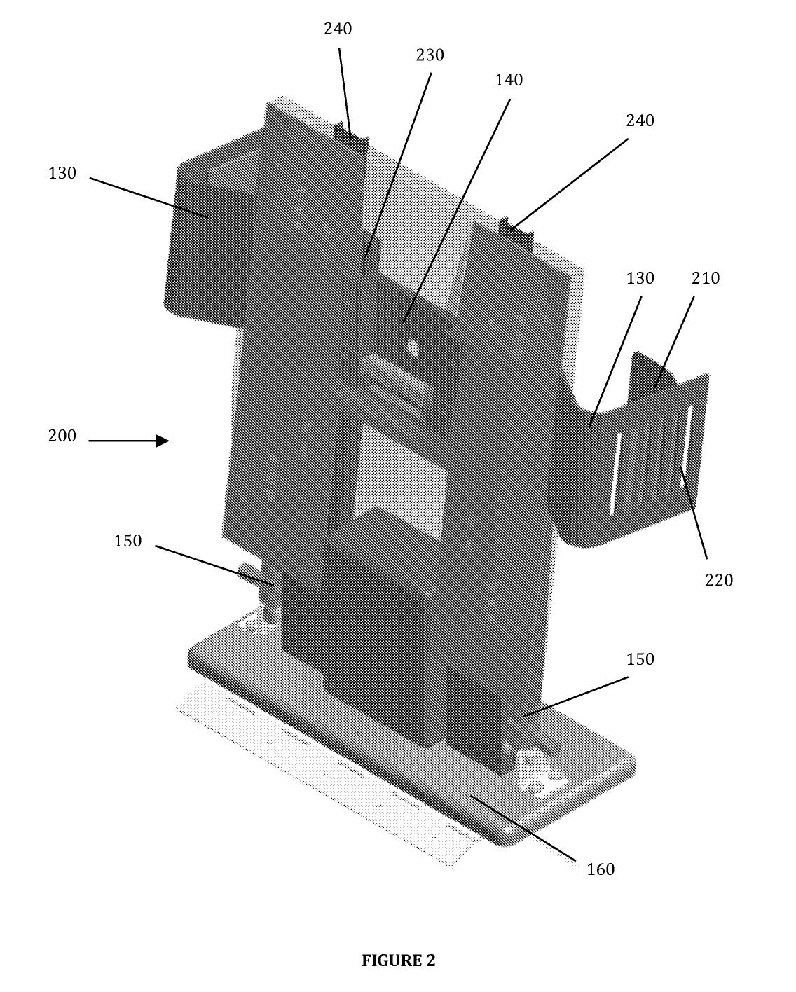

FIG. 2 is a perspective view of some of posture assistance stand internal elements 200 of the exemplary embodiment of the posture assistance stand shown in FIG. 1. Posture assistance stand internal elements 200 include clips 130 for attaching the posture assistance stand to a seat back surface of a vehicle seat. Clips 130 each include cupping part 210 that is adjustably coupled to adjustment element 220. Cupping part 210 may be plastic, metal part or any other appropriate material, and may be curved or angled to extend around a back side of a seat back surface. Each cupping part 210 may adjustably connect to a corresponding adjustment element 220 to enable a tight friction fit between the parts of clips 130 and the back side of a seat back surface to be formed, to thereby firmly hold the posture assistance stand in place on a seat. Base 160 of posture assistance stand internal elements 200 may rest against a seat bottom surface of a seat. Female adapter 140 may be rigidly attached to sliding element 230 that is adapted to slide up and down along rails 240. Rails 240 may be rigidly attached to a frame of posture assistance stand internal elements 200. Pistons 150 may be coupled to base 160 on a bottom end and pivotally coupled to sliding element 230 on a top end. In this manner, pistons 150 may operate to move sliding element 230 and female adapter 140 up and down with respect to base 160.

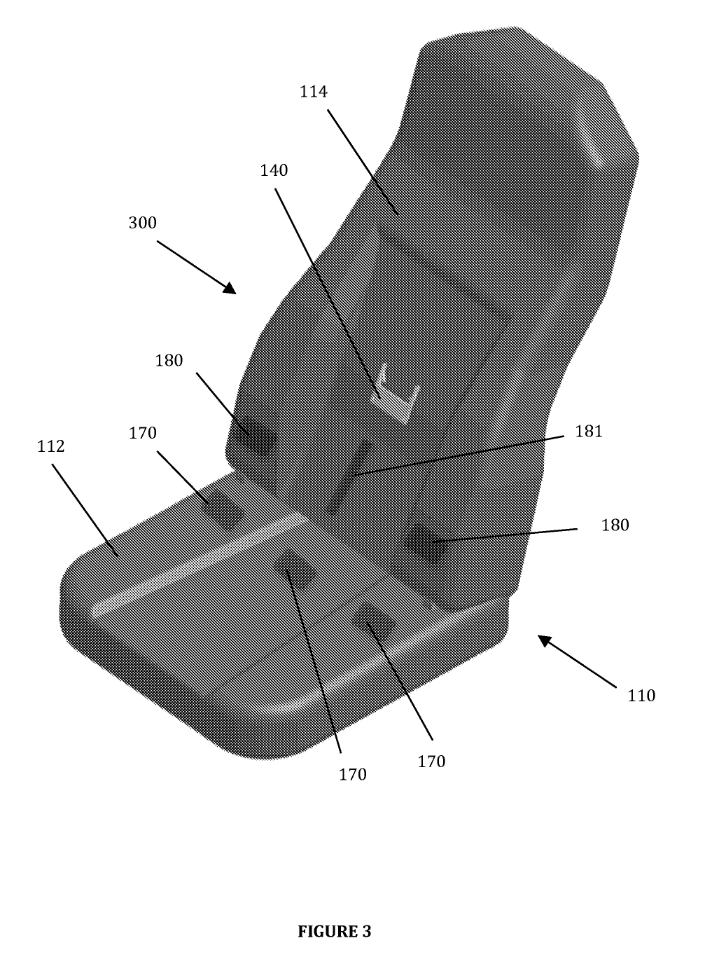

FIG. 3 is a perspective view of integrated posture assistance stand 300. Integrated posture assistance stand 300 includes vehicle seat 110, which includes seat bottom surface 112 and seat back surface 114. Seat bottom surface 112 may include one or more gluteal pressure sensors 170, and seat back surface 114 may include one or more back pressure sensors 180 and one or more flex sensors 181. On or above a lumbar region of seat back surface 114 may be one or more adapter receivers 140, which may operate to removably attach a posture feedback belt 500 worn by a user.

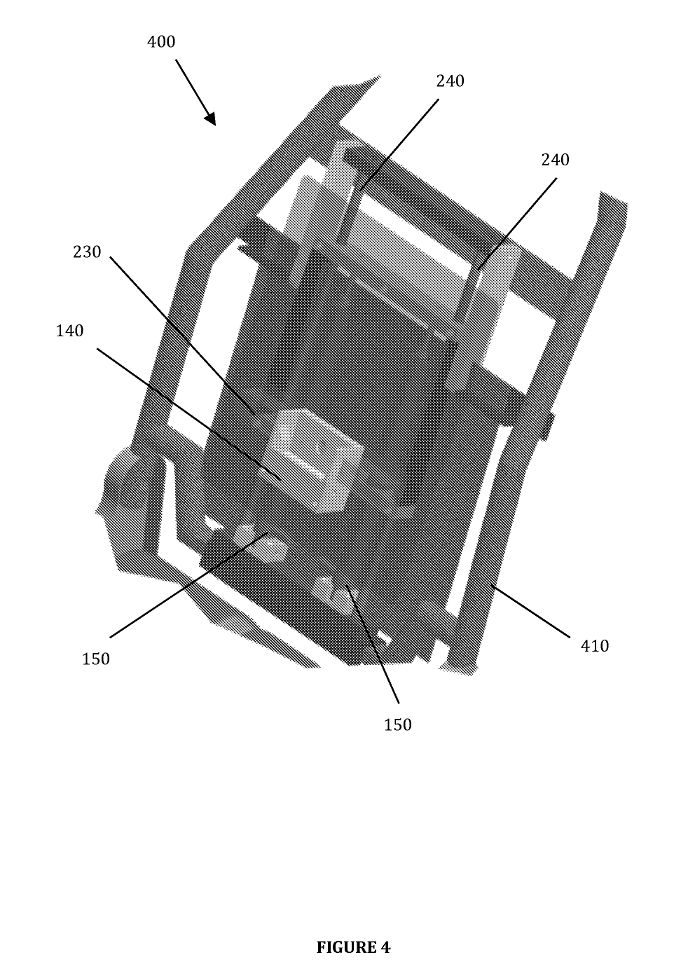

FIG. 4 is a perspective view of integrated posture assistance stand internal elements 400 of the exemplary embodiment of the present invention shown in FIG. 3. Integrated posture assistance stand internal elements 400 include female adapter 140, which may operate to removably attach a posture feedback belt 500 worn by a user. Integrated posture assistance stand internal elements 400 include frame 410, which may be a conventional seat frame, or may be customized for the purpose of attaching other elements of integrated posture assistance stand internal elements 400 to it. Frame 410 may be rigidly attached to a frame of the vehicle, and/or may be hingedly attached to a seat bottom frame to enable the seat to recline. Female adapter 140 may be rigidly attached to sliding element 230 that is adapted to slide up and down along rails 240. Rails 240 may be rigidly attached to frame 410 of integrated posture assistance stand internal elements 400. Pistons 150 may be coupled to frame 410 on a bottom end and to sliding element 130 on a top end, may be coupled to frame 410 on a top end and to sliding element 130 on a bottom end, or alternatively may be attached at both a top and a bottom end to frame 410, and to sliding element 130 on an intermediate portion that moves when pistons 150 are activated. In this manner, pistons 150 may operate to move female adapter 140 up and down with respect to frame 410.

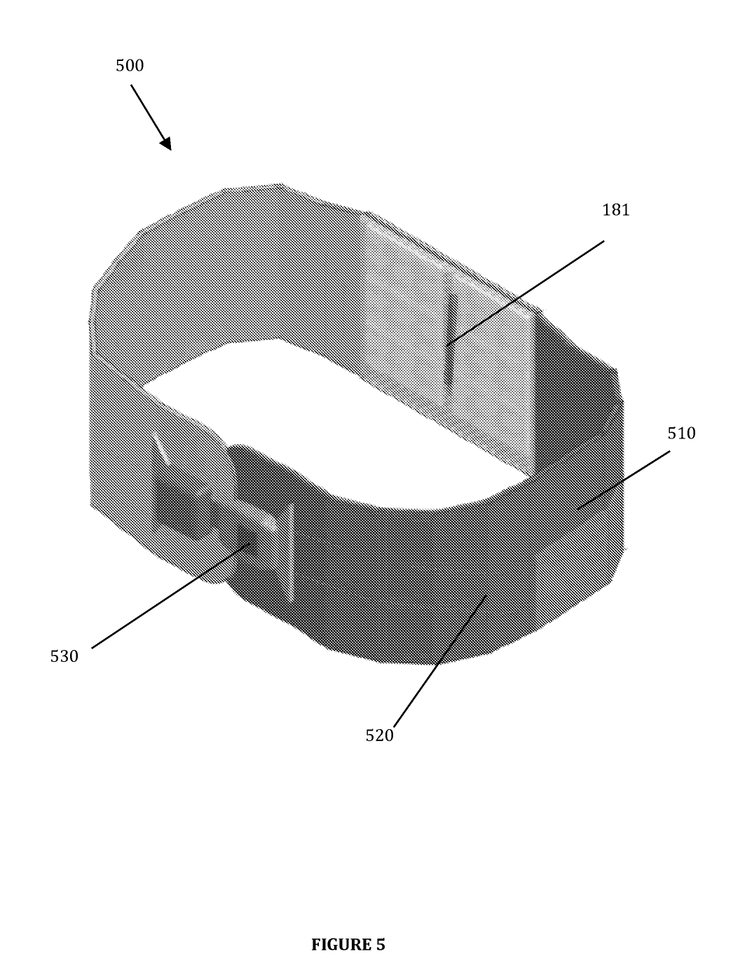

FIG. 5 is a front perspective view of posture feedback belt 500. Posture feedback belt 500 includes waistband 510, which may be neoprene, nylon, or any other appropriate material, and may be adjustably inflatable. Waistband 510 may open and close with buckle 530, or may close with Velcro and/or any appropriate method. Waistband 510 may be coupled to lumbar section 520, which may be neoprene, nylon, or any other appropriate material, and may be adjustably inflatable. Waistband 510 may be adapted to create a tight friction fit around the torso of a user. Waistband 510 may include internal lap belt 520, which may include a conventional seat belt or safety belt, and may include nylon or any other appropriate material. Lap belt 520 may close using the same method for closing waistband 510, and/or may close using buckle 530. Posture feedback belt 500 may include one or more flex sensors 181 and/or one or more pressure sensors.

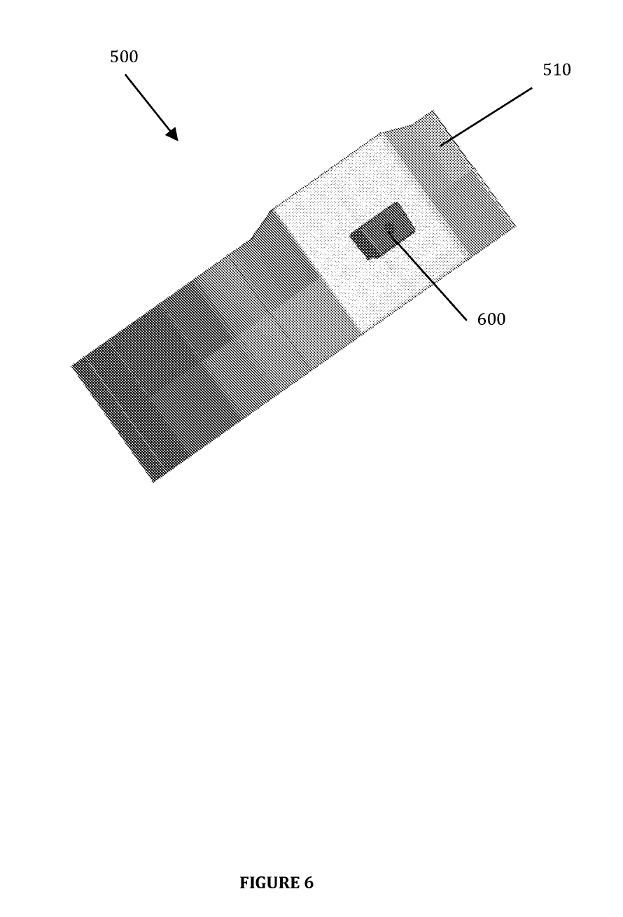

FIG. 6 is a rear perspective view of posture feedback belt 500. On a back side of posture feedback belt 500 may be positioned male adapter 600, which may be adapted to cooperate with a female adapter 140, in either an integrated or an add-on posture assistance stand as shown in FIGS. 1, 2, 3, and 4. Alternatively, male adapter 600 may be a female adapter adapted to cooperate with a male adapter in either an integrated or an add-on posture assistance stand (as shown in FIGS. 1, 2, 3, and 4). Male adapter 600 may include magnets or electromagnets of an opposite charge as the cooperating adapter of the integrated or add-on posture assistance stand, or may include any other appropriate locking mechanism adapted to cooperate with a female adapter to hold posture feedback belt 500 to an integrated or add-on posture assistance stand. Male adapter 600 may be controlled remotely or locally, manually or automatically, and may be responsive to an electronic stability program of a vehicle, an accelerometer, a collision sensor, or any other appropriate sensor to enable the locking and releasing of male adapter 600 from a female adapter receiver in either an integrated or an add-on posture assistance stand. In this manner, posture feedback belt 500 including internal lap belt 520 may function as a safety belt, thereby enabling the present invention to increase the safety as well as the comfort of a driver of a vehicle having the system.

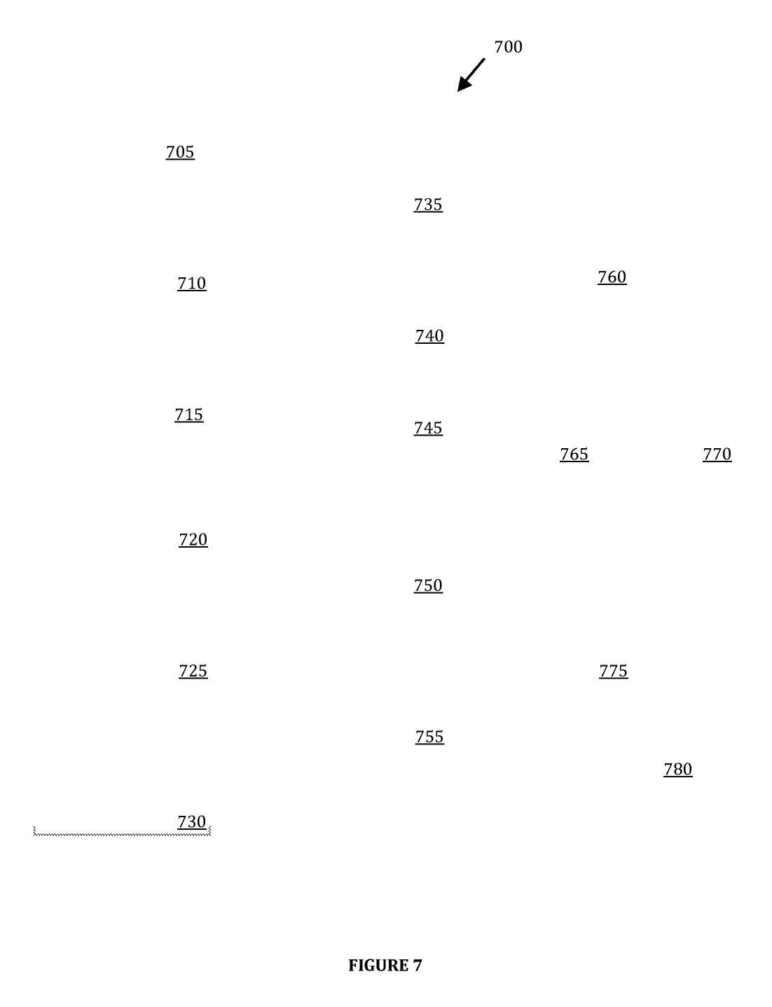

FIG. 7 illustrates method 700 according to an exemplary embodiment. Method 700 starts at start oval 705 and proceeds to operation 710, which indicates that the user enters the device by sitting against the belt. From operation 710, the flow in method 700 proceeds to operation 715, which indicates that the user adjusts and buckles the belt. From operation 715, the flow in method 700 proceeds to operation 720, which indicates that the lumbar support inflates with air. From operation 720, the flow in method 700 proceeds to operation 725, which indicates that the lumbar support rises vertically, unloading weight from user's torso. From operation 725, the flow in method 700 proceeds to operation 730, which indicates that the device prompts the user to sit in proper posture and records the posture setting of user. From operation 730, the flow in method 700 proceeds to operation 735, which indicates that the user sits with proper posture. From operation 735, the flow in method 700 proceeds to operation 740, which indicates that the lumbar support cycles the amount of support. From operation 740, the flow in method 700 proceeds to decision 745 if the user remains in the device. Decision 745 asks whether the user is slouched. If the answer to decision 745 is affirmative, the flow in method 700 proceeds to operation 750, which indicates that the feedback system is activated. From operation 750, the flow in method 700 proceeds to operation 755, which indicates that the user reassumes proper posture, and the system records the posture setting of user. From operation 755, the flow in method 700 proceeds to back to operation 735. If the answer to decision 745 is negative, the flow in method 700 proceeds back to operation 735.

From operation 740, the flow in method 700 proceeds to decision 760 if the user exits the device. Decision 760 asks whether the user wants to leave the belt on. If the answer to decision 760 is negative, the flow in method 700 proceeds to operation 765, which indicates that the user unbuckles the belt and leaves it with the device. From operation 765, the flow in method 700 proceeds to operation 775, which indicates that the lumbar support returns to the starting position. From operation 775, the flow in method 700 proceeds to end oval 780. If the answer to decision 760 is affirmative, the flow in method 700 proceeds to operation 770, which indicates that the user leaves belt on and steps away from device. From operation 770, the flow in method 700 proceeds to operation 775.

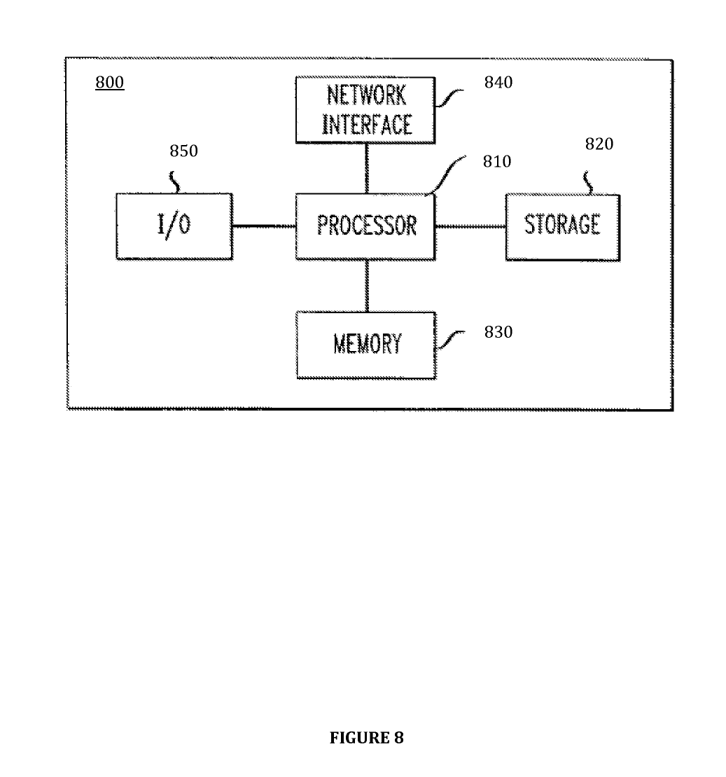

FIG. 8 illustrates a computer system according to an exemplary embodiment. Computer 800 can, for example, operate pistons 150 of posture assistance stand 100 and/or integrated posture assistance stand 300, may operate air bladders or tensioning elements of posture feedback belt 500, and/or may operate one or both of female adapter 140 and male adapter 600. Additionally, computer 800 can perform the steps described above (e.g., with respect to FIG. 7). Computer 800 contains processor 810 which controls the operation of computer 800 by executing computer program instructions which define such operation, and which may be stored on a computer-readable recording medium. The computer program instructions may be stored in storage 820 (e.g., a magnetic disk, a database) and loaded into memory 830 when execution of the computer program instructions is desired. Thus, the computer operation will be defined by computer program instructions stored in memory 830 and/or storage 820 and computer 800 will be controlled by processor 810 executing the computer program instructions. Computer 800 also includes one or more network interfaces 840 for communicating with other devices, for example other computers, servers, or websites. Network interface 840 may, for example, be a local network, a wireless network, an intranet, or the Internet. Computer 800 also includes input/output 850, which represents devices which allow for user interaction with the computer 800 (e.g., display, keyboard, mouse, speakers, buttons, webcams, etc.). One skilled in the art will recognize that an implementation of an actual computer will contain other components as well, and that FIG. 8 is a high level representation of some of the components of such a computer for illustrative purposes.

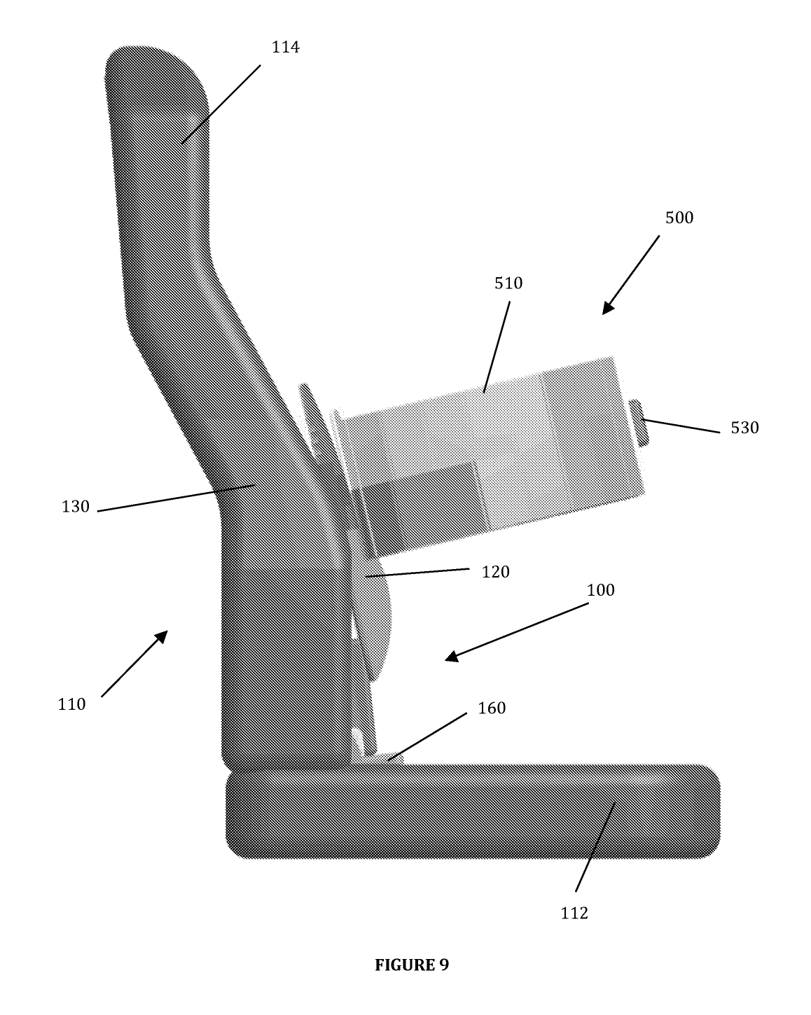

FIG. 9 is a side view of an exemplary embodiment of posture assistance stand 100 attached to vehicle seat 110 with detachable posture feedback belt 500 attached to posture assistance stand 100. Alternatively, posture assistance stand 100 and posture assistance stand 100 may be used with a seat that is not in a vehicle. Posture assistance stand 100 attaches via clips 130 to seat back surface 114 of seat 110. Clips 130 may include a plastic or metal part that may be curved or angled that may extend around a back side of seat back surface 114, and which may adjustably connect to posture assistance stand 100 to enable a tight friction fit between the parts of clips 130 and the back side of seat back surface 114 to be formed, to firmly hold posture assistance stand 100 in place on seat 110. Clips 130 may additionally or alternatively include a strap extending around a back side of seat back surface 114, may include Velcro, may attach to an anchor point on seat back surface 114, or may attach by any other appropriate method. Base 160 of posture assistance stand 100 may rest against seat bottom surface 112 of seat 100 or may wedge between seat back surface 114 and seat bottom surface 112. Base 160 may alternatively attach to seat bottom surface 112 using a strap extending around a bottom side of seat bottom surface 112, may include Velcro, may attach to an anchor point on seat bottom surface 112, or may attach by any other appropriate method. Posture assistance stand 100 may include a lumbar support 120, which may include foam, nylon, neoprene or any other appropriate material. Lumbar support 120 may also be adjustably inflatable. Lumbar support 120 may be designed to rest against a lumbar area of a user when the posture assistance stand 100 is attached to seat 110 and the user sits in seat 110. Lumbar support 120 may include a female adapter or alternatively a male adapter. The female or male adapter may include magnets, electromagnets, or any other appropriate locking pieces adapted to cooperate with an adapter to connect to a detachable posture feedback belt 500.

Detachable posture feedback belt 500 may include waistband 510, which may be neoprene, nylon, or any other appropriate material, and may be adjustably inflatable. Waistband 510 may open and close with buckle 530, or alternatively may close with Velcro and/or any appropriate method. Waistband 510 may be coupled to a lumbar section, which may be neoprene, nylon, or any other appropriate material, and may be adjustably inflatable. Waistband 510 may be adapted to create a tight friction fit around the torso of a user. Waistband 510 may include an internal lap belt, which may include a conventional seat belt or safety belt, and may include nylon or any other appropriate material. The lap belt may close using the same method for closing waistband 510, and/or may close using buckle 530. Posture feedback belt 500 may include one or more flex sensors and/or one or more pressure sensors.

In order to connect posture feedback belt 500 to posture assistance stand 100, a male adaptor on posture feedback belt 500, or alternatively on posture assistance stand 100, should be attached to a female adaptor on posture assistance stand 100, or alternatively on posture feedback belt 500. FIG. 9 demonstrates use of an exemplary embodiment of the present invention on vehicle seat 110, which may be a truck seat, but the present invention may alternatively be used at home or at an office. Posture feedback belt 500 may wrap around the waist of a user and, when detached from posture assistance stand 100, may provide feedback in the form of sound/vibration when the user lifts with a posture that may cause injury. Posture feedback belt 500 may initially record the user's standing posture through a flex sensor located in the rear of the belt. Once posture feedback belt 500 records the user's posture, the user can adjust the sensitivity of posture feedback belt 500 in order to determine at what lifting posture position feedback should be provided. The sensitivity setting enables posture feedback belt 500 to be customized for different users having different movement requirements, and different individuals may have a different range of movement before entering a high-risk lifting posture. Once the user deviates beyond the set sensitivity range, posture feedback belt 500 provides a sound/vibration to alert the user that they are in a high-risk lifting position.

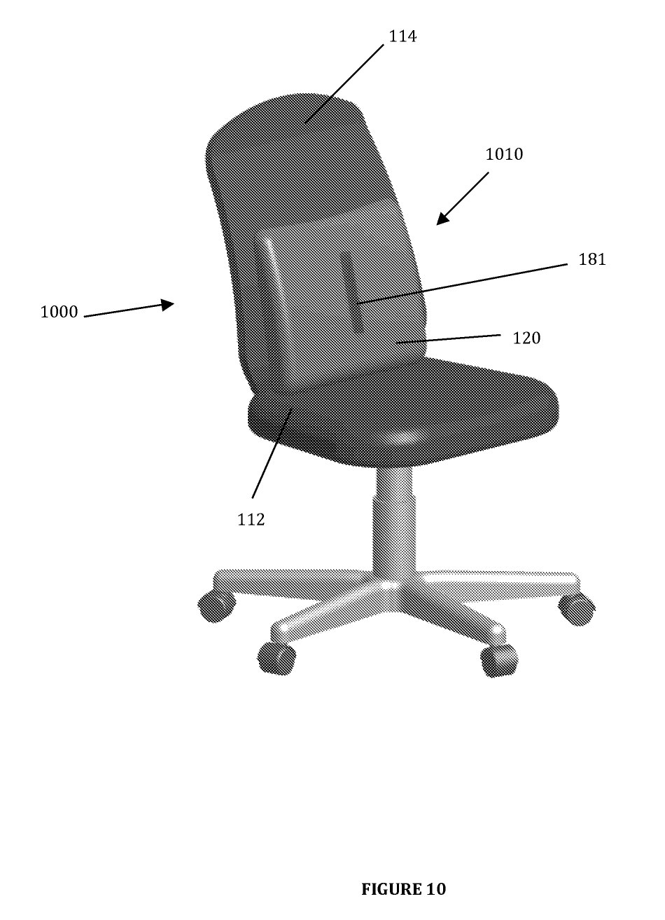

FIG. 10 illustrates office chair 1000 according to an exemplary embodiment of the present invention. Office chair 1000 provides posture feedback through lumbar support posture assistance system 1010. Lumbar support posture assistance system 1010 does not include a belt, but instead includes only lumbar support 120. Lumbar support posture assistance system 1010 provides posture feedback to a user in an office, home, automobile, or truck, and encourages sitting with active posture by providing feedback in the form of sound and/or vibration when the user sits with a slouched posture. Sitting with an active posture activates core musculature and facilitates caloric expenditures. Initially, lumbar support posture assistance system 1010 records the user's sitting posture through flex sensor 181 located in the center of lumbar support 120. Alternatively, flex sensor 181 may include a plurality of flex sensors, which may be located in any particular locations in lumbar support 120, seat back surface 114, and/or seat bottom surface 112. Once lumbar support posture assistance system 1010 records a user's posture, the user can adjust the sensitivity of the device in order to determine at what posture position feedback should be provided. Once the user deviates beyond the set sensitivity range, lumbar support 120 will provide a sound and/or vibration to alert the user that they are in a slouched position. A higher sensitivity setting will activate the feedback upon a minor slouch, whereas a less sensitive setting will allow a user to slouch more before the feedback is activated.

The invention disclosed herein may include a system and method for relieving lower back pain by unloading weight from the user's back while simultaneously monitoring the user's posture by recording an ideal “baseline posture” and providing feedback to the user in the form of audio, visual and/or tactile signals when the user demonstrates unsatisfactory posture, determined by a preset variance from the adjustable baseline posture.

The present invention provides a method and apparatus for monitoring driver alertness that utilizes a posture feedback belt mounted touch detecting (flex) sensors to measure the driver's posture initially and record the driver's ideal baseline posture in order to alert the driver when they begin to slouch or become drowsy and thus deviate from their ideal posture.

Also disclosed is a method for providing a wake-up mechanism for drivers who begin to fall asleep while driving. A person in a seated position who begins to fall asleep loses proper posture. Thus, the posture feedback system may also serve as a wake-up mechanism for drivers who are drowsy and begin to lose their posture.

An embodiment of the present invention may include one or more of several possible components: a posture feedback belt which may have an inflatable air bladder(s); one or more touch detecting (flex) sensors affixed to the posture feedback belt in the lumber region of a user's back; one or more touch detecting sensors in other areas of the posture feedback belt; an adapter on the back side of the posture feedback belt used to attach to a lumbar support; a posture support stand with air pistons, hydraulics, a motor, a spring driven by a motor, or actuators that may be integrated within a seat or may be an accessory adapted to be attached to a seat; a lumbar support, which may contain an inflatable air bladder(s) located on vertical support rails of the posture support stand; and/or another adapter located on a front of the lumbar support. The adapter on the back of the posture feedback belt may connect to the adapter on the front side of the lumbar support, and the posture support stand may provide power and/or air to the posture feedback belt via the adapter connection.

A first-time user of the invention may first put on the posture feedback belt and adjust its size. The user then sits on a seat and against the seat back that has the posture support stand located on it. The user sits against the lumbar support and aligns the adapter on the back of the posture feedback belt to the adapter on the front side of the lumbar support. The adapter connection may provide power and air to the posture feedback belt. The posture support stand, once the user is wearing the posture feedback belt and is connected to the lumbar support via the adapters, may provide an upward force by elevating the lumbar support, and in turn reduce the amount of pressure on a user's low back. The weight reduction provided by the posture support stand may be capped to be no greater than 30 lbs. of force in order to prevent atrophy of the user's muscles. This support may assist the user to sit properly with healthy, active posture for long periods of time.

The user may have the option of entering a baseline threshold posture manually or in response to a voice or other audible instruction from the system. In the case of a voice instruction, the system may inform the user to sit with their preferred active posture. After several seconds, the system records this posture by recognizing the amount of weight the user has placed on the pressure sensors or the curvature of the flex sensor(s) and records it as the baseline posture. Deviation above or below the baseline posture beyond a preset or adjustable amount may put reduced or additional force or pressure on one or more of the pressure sensors, or may cause the flex sensor(s) to bend additionally, thus activating the feedback system. The user may also have the option of choosing the effect utilized by the feedback system, for instance vibration, sound or a combination of both. Programming of the baseline posture may be done through the control panel or through an audio output that may prompt the user to sit properly and the system may record a baseline posture. The user may also have the option to modulate the pressure sensing components, thus deciding which pressure sensors to use. Slouching into kyphosis, defined as an abnormal, convex curvature of the spine, with its distinct pressure profile, may be interpreted as a deviation from lordosis, which is defined as sitting erectly with an adequate degree of forward curvature of the spine in the lumbar region. Such a deviation would trigger the effecter feedback system.

Several types of slouching have been identified, including a torso slouch and a hip slouch. In the torso slouch situation, the user leans forward and thus places additional weight on the seat portion. When one or more of the pressure/flex sensors determines that an increase of pressure has occurred, the system may provide feedback to the user indicating that the user should adjust their posture to the baseline position. In the case of hip slouching, the user slides their hips forward and puts additional weight on the pressure sensor in the lumbar support, which also may activate the effecter feedback. The pressure sensors, however, are not limited to picking up these two forms of slouch and could potentially alert the user to any posture that significantly deviates from their ideal posture.

The adapter connection may be designed so as to permit quick release from the posture support stand. When a user wearing the posture feedback belt disconnects from the posture support stand by forward force, the posture feedback belt may remain around the user's waist and function as a back brace. Furthermore, the posture feedback belt may provide support so as to prevent strain during heavy lifting. Support may also be provided from the air that remains in the posture feedback belt after disconnecting from the receiver stand. Additionally, the one or more pressure and/or flex sensors may continue to operate via batteries or another mechanism when the user is wearing the air harness separated from the receiver stand, thus providing feedback while the user is mobile.

Through a control panel, the user may customize and set one or more of the following: the level of inflation for the posture feedback belt and/or lumbar support; the amount of upward force provided by the lumbar support; the baseline posture from which feedback may be provided; the type of feedback desired (sound or vibration); the desired sensitivity of the feedback system; whether the system should automatically adjust and/or vary the amount of inflation for the posture feedback belt and/or lumbar support; and whether the system should automatically adjust and/or vary the amount of upward force provided by the posture support stand. The user may select the sensitivity of the feedback system, so that minor deviations in posture will not activate the effecter feedback. On the other hand, a user who wants an immediate response to deviations in posture may select a more sensitive feedback so that a slight decrease from baseline posture can activate the effecter feedback.

The invention may also have the feature of automatically adjusting the posture support provided based on the posture of the individual. If the user begins to supplement the upward force with other methods of unloading the lower spine, such as excessive reclining or pushing down on arm rests, the system may reduce the amount of upward force provided, in order to avoid creating a crutch effect. Therefore, the user may be prevented from becoming reliant on the upward force provided and instead will be required to condition their musculature in order to improve their posture. Additionally or alternatively, if the user demonstrates poor posture that activates the feedback system for a long time or continues to activate the feedback system several times within a certain time frame, the system may increase the amount of upward force provided in order to assist the user, who may be struggling.

An example of the crutch effect, as described above, is when drivers recline in their seats. Excessive reclining while driving or working is an unhealthy way of unloading weight from the lower back, as it tends to strain the neck and promotes sleepiness. However, a small degree of recline is acceptable. Using a gluteal pressure sensor, the invention may reduce the amount of upward force provided according to the amount of unloading resulting from reclining. If the average amount of pressure exerted on the gluteal pressure sensor decreases, the amount of upward force provided from the device may decrease in order to keep the user's muscles engaged and to prevent the crutch effect.

Another example of the crutch effect is when a driver pushes down on a vehicle's arm rests to ease the burden on his or her lower back. Excessive use of armrests is an unhealthy way of unloading the lower back as it irritates elbows, restricts breathing, increases blood pressure and strains the neck. A small degree of armrest use however, is acceptable. Using a gluteal pressure sensor, the invention may adjust the amount of upward force provided according to the amount of unloading resulting from transferring torso weight onto the armrests. When a driver places a great amount of weight on the armrest, the average amount of pressure exerted on the gluteal pressure sensor decreases, and the amount of upward force provided from the device may decrease.

Obese drivers may place a great deal of weight on their lower back and may be more susceptible to back pain. Obese drivers may require greater degrees of upward force to feel the same pain relief as non-obese individuals, and may also require more help to sit with proper posture. An obese driver placing greater weight on the gluteal pressure sensor may cause the system to automatically increase the amount of upward force provided.

The posture feedback system according to the present disclosure may automatically adjust the amount of posture support with a cycling function to increase long-term comfort and to minimize the operational difficulties for the user.

The posture feedback belt may further include elastic, neoprene, or semi-rigid material that can fit around a user's waist. The posture feedback belt element may include an air bladder or a plurality of air bladders and may include a closing mechanism, for example a buckle or velcro. In other alternatives, the posture feedback belt may not include air bladders but instead may include bladders holding another fluid adapted to exert pressure. The posture feedback belt may further include one or more flex sensors affixed to the belt's interior in a location that will make contact with a user's lumbar region. The posture feedback belt may also include a male adapter located on its rear side, which may receive electricity and/or air pressure by connecting to a female adaptor on the front of the lumbar support. The male and female adaptors may contain magnets, electromagnets or a funneling mechanism that allows the user to connect and disconnect from the lumbar support without using their hands. The preferred adapter design according to the present invention makes it convenient and easy for the user to attach and detach from the posture support stand, even while driving.

An alternative embodiment may contain the posture feedback belt as affixed or attachable to the lumbar support via velcro, buckles or some other mechanism, and thus would not require the male and female adapters.

A pressure source may be included in a system according to the present invention for inflating or deflating the air bladder, and/or providing pressure to air pistons that may provide an upward force to the belt. The pressure source may be an external compressor or may use the pressure source found in a truck.

Although the user may want to initially customize their preferences, the apparatus may offer a default option to operate automatically with the user's preset selections, after the posture feedback belt is engaged with the posture support stand and buckled around the user's waist. First, the posture feedback belt may inflate to the desired preset level from pressure generated from the pressure source, at which point a valve, through an electrical and/or mechanical mechanism, may close. The valve may redirect air pressure to the air piston(s), which may be connected to the pressure source via conduit. Next, the pressure source may increase air pressure to the pistons, through the opening of the valve, which may cause the lumbar support, and the posture feedback belt to rise.

The exemplary combination of a posture support device providing upward force for sitting and a mechanism that discourages behavioral and mechanical dependence using feedback provides the advantage of providing pain relief and conditioning proper posture. A posture feedback system can significantly reduce the amount of upward force that a user needs over time by encouraging proper posture causing a user to strengthen their support muscles. In this manner, the user may ultimately rely less on the traction force provided by the system. Additionally, the upward force conversion can make the exercise of good posture more sustainable over time. By permitting long duration use, the feedback system can also be used as a rapid and reliable method of detecting sporadic sleepiness of users, such as commercial drivers.

The unique combination of posture support and posture feedback avoids the user becoming reliant upon the posture support because the posture feedback element ensures that the user sits with proper posture. It further encourages users to employ their core musculature by sitting with an active, healthy posture, which results in greater caloric expenditures. Without the posture feedback to contribute to good posture, the user will naturally use the posture support stand as a crutch and thus will relax their support muscles to transfer increasing amounts of weight to the device. The continuous relaxation of support muscles during prolonged periods may atrophy the support muscles that must be well exercised to protect the spine, and paradoxically to reduce painful spasms. Continuous traction devices for sitting also de-condition the behavioral component of good posture, and when a user sits in a normal chair without the support, he or she may feel even less inclined to sit properly than before. Well before pain reaches conscious levels in the patient, these devices may influence the patient's mood/clarity, and the increasing forces exerted on the abdominal and chest wall through an ever-increasing crutch effect may also have the long term effects of deforming the rib cage, reducing blood flow to the abdominal wall, and/or increasing intra-abdominal pressures to the point of encouraging hernia formation. The instinctual transfer of upper body weight onto the traction device may continue until eventually the traction apparatus become a source of pain.

A conduit may be provided for transferring pressure between the pressure source and the air harness and/or the air pistons. A receiver stand may be provided for connecting to the air harness and for providing upward force. Vertical support rails may be attached to the receiver stand base, and a lumbar support may be attached to the vertical support rails, whereby the lumbar support may rise vertically. An adapter receiver connected to and located on the front side of the lumbar support may connect to the adapter. A female electric/pressure connection may transfer electricity and air or fluid pressure to a male electric pressure connector. Air pistons may be attached to the receiver stand and may provide upward force which increases as the distance between the air harness and the seat increases, thereby providing the user with assistance in the form of weight relief. Programmable circuitry and a control panel may enable entry of user-adjustable parameters. A speaker and/or a vibration mechanism may provide posture related feedback.

The lumbar support may contain an auto-coiling band of elastic material beginning at the base of the lumbar support and extending below the lumbar support. The air harness may rise slightly on the torsos of certain individuals, and the elastic material can prevent the upward pull by providing some downwards pull. Furthermore, the elastic material may coil upwards.

The apparatus may contain a cushion with a pressure sensor located on the cushion in order to measure changes in pressure. This pressure sensor may be used to provide posture related feedback in conjunction with the pressure sensor located in the lumbar support. The pressure sensor may be a direct pressure sensor, or alternatively or additionally may be a touch sensor (also referred to herein as a flex sensor), which may measure the amount of bend of the sensor body.

In another embodiment, the male adapter and female adapter may be designed in a funnel shape, or alternatively in a ball and socket shape, so as to permit ease of connectivity.

In another embodiment, the apparatus may include a check valve adapted to protect against inappropriate exiting of air into the system from the posture feedback belt after it has been disengaged from the posture support stand. The system may also contain safety release valves, which ensure that the air bladders do not inflate beyond a certain pressure. The apparatus may additionally be equipped with a valve adapted for adjustably limiting a rate of air in the conduit during inflation.

In another embodiment, the apparatus may include one or more solenoid valves. This embodiment is particularly important for enabling the automatic adjustability of the system as described above. The programmable circuitry may regulate the flow of air from the pressure source by opening one of the solenoid valves allowing air to flow to the lumbar air harness, and upon reaching the user-adjusted parameter, closing that valve and opening another solenoid valve allowing air to flow to the air pistons. The programmable circuitry may control operation of the pressure source and independently control the solenoid valves by means of user-adjustable parameters.

In another embodiment, the posture feedback belt may contain a cooling mechanism to enable long periods of use without excess heat built up. As a constant pressure connection is available to the posture feedback belt while it is engaged to the receiver stand, this air pressure may be used to ventilate the posture feedback belt's interface with the user. Prior to ventilation against the user, the air could potentially be run through a cooling system, which could include of a bag of liquid/gel (potentially cooling in refrigerator like a cold pack, or filling with ice water, etc.) that surrounds coils of tubing delivering “leak air” to leak holes in the air harness. The “cold pack” could not only be designed to remove heat energy from the air, but also dissipate its own accumulated heat through nearby heat conducting pathways on the receiver stand. In this embodiment, an air conduit can run through the center of the posture feedback belt, between the air bladders. Air can be diverted through coiled tubes that run through a reservoir of cool water located in the rear of the posture feedback belt. Subsequently, the cooled air may inflate the air conduit. As the air conduit inflates, it creates a new point of skin compression, which allows the cool air to vent throughout the air harness as well as through perforations on the outer material of the air harness, thus cooling the user. In another variation of this system, the cold-water reservoir could be placed within the lumbar support.

Another variation of a cooling mechanism may include a thermocouple cooling system that could use electrical energy to cool the air before it reaches the posture feedback belt, or something of the like.

In another embodiment, the air harness may include the lumbar support may be attached to the vertical support rails, and may contain the adapter receiver on its front side. In this embodiment, the air harness may contain the adapter on its backside, and the pressure sensor may be located within the lumbar support.

In another embodiment, the air pistons may be substituted for springs, a ladder notch system or any other electric, mechanical or electro-mechanical method of raising the apparatus to achieve upward force.

In another embodiment, the pressure source may be an electric pump, or the pressure source found under most truck seats.

In another embodiment, the buckle may be a strap, clasp, Velcro or other fastening mechanism.

Also disclosed is a method for weight loss and caloric reduction. Sitting with proper posture is more effective in reducing calories than sitting in a slouched position. Thus, through the use of this invention, users will be able to reduce more calories, which may result in weight loss, due to sitting with proper posture.

The invention may be an independent portable model, or may come integrated in a seat. In the integrated model, the receiver stand may be located behind the seat back's upholstery. In the independent model, the receiver stand fits between the seat back and the seat.

The device may be integrated to a truck seat and the existing pressure source found in most trucks may provide pressure to the air pistons.

The air leak/cooling system may include a cold air reservoir located in the rear of the air harness, and a venting/air leak conduit that runs throughout the length of the air harness and through its center between the air bladders. The cold-water reservoir may be filled via an inlet/outlet that may be tucked into the air harness. The venting/air leak conduit may inflate when the solenoid valve in the air harness vents air. The air may pass through tubing through the reservoir of cold water and subsequently cool before filling the venting/air leak conduit. Once the venting/air leak conduit is full, it may provide a new point of skin compression around the user's waist, which may cool the user. The venting/air leak conduit may then release the cooled air through leak holes, which may cool the inner part of the air harness and thus the user. The cooled air may circulate through the interior part of the air harness, and then may exit the air harness through breathable material on the interior side of the belt, thus providing cool air on the user's waist region.

The lumbar support may be affixed to a metal plate to provide upward force via the air pistons and the pressure source. The buckle may be substituted with Velcro, a clasp or any other fastening mechanism.

The female buckle may contain an apparatus that detects the connection of the male buckle to the female buckle, which may communicate with the valve system. In this embodiment, when the buckle is closed, the circuit is completed and the posture feedback belt can receive power and pressure when connected to the stand. In this embodiment, when the posture feedback belt is connected to the posture support stand, and the user opens the buckle, the air pistons and posture feedback belt subsequently vent.

The lumbar support may be included of any substantially static materials, moldable materials, flexible fabrics containing elastic, and/or any other fabrics materials. In one embodiment the lumbar support may contain an airbag or plurality or airbags that inflate behind a user's lower back/thorax/sacrum.

In one embodiment, the lumbar support may be affixed to the waist harness and will contain the adapter on its rear side. In this embodiment, the lumbar support may connect directly to the stand, which can contain the adapter receiver and the female electric/pressure connector.

In another embodiment, the lumbar support may contain a band of elastic material.

The band of the posture feedback belt may contain an opening in the bottom which allows passage of a standard safety seatbelt. In such an embodiment, the seat belt buckle may pass through another buckle, enabling the user to buckle the seatbelt and simultaneously activate the device.

In another embodiment, the posture feedback belt may contain an internal rigid belt that passes through the lower region of it, in order to disperse the force of a vehicle collision. In this embodiment, the adapter mechanism may restrain the user against the seat back in case of a collision or if the driver is driving above a certain speed. The internal rigid belt within the posture feedback belt will provide a restraint for the user.

In an embodiment, the waist harness may contain a cooling mechanism, which may ventilate the waist harness from time to time. This feature is meant to address a potential problem whereby the waist harness may get warm around a user's waist. A water reservoir may be contained in the lumbar support with conduits for air to pass through. In this system, the air may pass through the cooled tubes and may fill an air bladder in the center of the waist harness (between the other air bladders). This cooled air bladder may allow the cooled air to pass through small perforations in the bladder and through perforations on the interior of the belt. Thus, the cooled air would cool the interior of the belt, and would eventually flow over the user's skin. Other potential systems may include thermocouple cooling system that could use electrical energy or cold metal pipes to cool the air before it reaches the waist harness, or any alternative appropriate cooling mechanism.

The posture feedback belt may be made of neoprene or another elastic material, for instance memory foam, another type of padding, and/or gel cushioning on the interior. This will help contour the belt around the user's waist. The posture feedback belt may contain a pressure release button to allow the venting of air if the waist harness is not connected to the stand, yet is still inflated.

In one embodiment, the posture feedback belt may contain a plurality of airbags, which may inflate and provide the user with air pressure around its waist, contouring to that user's waist. The posture feedback belt may include a check valve to protect against inappropriate exiting of air from the waist harness after the user has disengaged from the stand. The posture feedback belt may contain safety release valves to ensure that the air may not inflate beyond a certain level. The waist harness may contain a pressure release button to release air from the waist harness when the user is detached from the stand.

The apparatus may contain a valve adapted for adjustably limiting a rate of air in the conduit during inflation. In one embodiment the lumbar support may be affixed to the waist harness and contain the adapter on the back end. Thus, the user would sit against the stand and when the user detaches from the device, he/she will rise wearing the waist harness with the lumbar attached to it. In another embodiment the waist harness may be attached to the stand. In this embodiment, the left arm flap that passes across the user's waist may contain an opening through which the seatbelt can pass. In this case, the seatbelt may nestle into another buckle and when the user attaches its seatbelt. The device may be activated by pressure or an electronic signal. This model may be more attractive for long-haul truckers who do not enter and exit the vehicle as often and who may wear their seatbelt more often.

The receiver stand may contain a cushion with a gluteal pressure sensor to measure changes in pressure from the user-preset baseline posture.

The pressure source may be an electric pump, or may connect to the pressure source found in most trucks under the truck seat.

The posture feedback belt may contain a pressure release button to allow the venting of air if the waist harness is not connected to the stand, yet is still inflated. The valve system may not contain the valve that provides air to the waist harness. In this case, the waist harness may contain that valve.

The device may contain a plurality of pressure sensors located throughout in order to measure changes in posture and provide posture-related feedback. The pressure sensors may continue to work while the user is mobile with the waist harness connected and inflated. This would enable a user to receive posture-related feedback while on the move.

The device may auto-adjust, providing more or less upward force, depending on whether the user's posture (as measured by pressure on the sensors) deviates beyond a baseline or preset threshold set automatically or by the user. A certain number of deviations may be required within a certain time frame to activate the auto-adjustment. The upward force in and of itself may create a “crutch effect” that a user begins to rely on. The addition of the posture feedback system reinforces positive behavior by requiring the user to sit up straight in order to avoid triggering the effector feedback. Additionally, the posture feedback mechanism may serve as a driver wake-up system, for example when the driver begins to nod-off and slouches. In this situation, the effecter feedback may be activated.

The device may lift from the sacrum/lower lumbar region, as opposed to merely lifting the thorax/chest area. Traction is coupled with posture related feedback in the system. The belt is may include of a semi-vertically rigid laterally flexible material. The belt has a sacral support which resists the counter torque imposed by the forward center-of-gravity from the body. This sacrum support helps to achieve proper traction all around and not only dragging up from behind, as might occur if no sacrum support were provided. The system may also have an adapter which provides a means of connecting to a mechanism for providing upwards force or traction.

Air pressure may be provided throughout the belt, gripping around the torso of a person with an electric and/or pressure connection through the adapter mechanism.

In some exemplary embodiments, only the gluteal sensor will affect the auto-adjustability of the device.

Continuous passive motion may be used in conjunction with the present invention by incorporating a continuously moving lumbar support, so the user is not in the same position for too long, and is always encouraged to use their muscles in making small movements. This may take the form of an air inflatable bag or bags within the lumbar support.

An internal compressor within the belt may be provided, thereby avoiding to have air or fluid pressure transferred through the adapter mechanism. In some exemplary embodiments, electric power and signaling may transfer from the posture support stand to the posture feedback belt. The electric power may activate a compressor in the belt which in turn may inflate the rear portion of the posture feedback belt. Then the motor on the stand may provide traction. These approaches for posture support, namely pneumatics and/or an electric motor, are therefore possible, alone or in combination.

An algorithm according to the present invention may be able to detect the user's body type and adjust the sensitivity levels of each pressure sensor according to that body type. The different body types may activate different sensors in different ways. Creating such an algorithm may allow the system to tailor the sensors according to the user's body. The user may be able to program the sensitivity of all of the sensors collectively. This algorithm may not affect the sensitivity of the feedback system as a whole, but may be used only for each individual sensor.

The user can either buckle the belt before entering the device by sitting against the lumbar, or can enter the device and then buckle it if belt is left with the device.

A model may be used in certain embodiments that uses one or more measurement signals as inputs and outputs one or more output signals, including for example a posture assistance amount and/or a feedback signal. The model may be adjustable based on a user's preference, including the adjustment of certain thresholds determining the type or amount of output signals based on a particular input signal or signals. Additionally, the model may be self-adjusting based on trends of an input signal or signals, or based on the immediate change in an input signal or signals following a particular output signal or signals, for instance in response to a feedback signal.

While only a limited number of preferred embodiments of the present invention have been disclosed for purposes of illustration, many modifications and variations could be made thereto. The present application is intended to cover all of those modifications and variations which fall within the scope of the present invention, as defined by the following claims.

We claim:

1. A device for monitoring a posture of a user, comprising:
a belt adapted to encircle a torso of the user and provide an upward force to at least part of a spinal region of the user in response to a signal from a controller;
at least one sensor adapted to provide at least one measurement signal to the controller; and
a feedback system activated by the controller to alert the user that a posture of the user is determined to be outside an acceptable range based on the at least one measurement signal.

2. The device of claim 1, wherein the feedback system signals to the user with at least one of a sound and a vibration.

3. The device of claim 1, wherein the posture of the user is determined to be outside the acceptable range when at least one of:
the at least one measurement signal exceeds a first predetermined threshold;
the at least one measurement signal falls below a second predetermined threshold; and
the at least one measurement signal is a plurality of measurement signals satisfying a preprogrammed model.

4. The device of claim 3, wherein, after the posture of the user is determined to be outside the acceptable range, at least one of the first predetermined threshold and the second predetermined threshold are changed based on an adjusted posture of the user.

5. The device of claim 4, wherein the adjusted posture of the user is identified based in part on a speed at which the users assumes the adjusted posture after the user is alerted by the feedback system.

6. The device of claim 1, wherein the controller is adapted to signal the belt to provide the upward force to the user when the controller determines that the posture of the user is outside the acceptable range.

7. The device of claim 1, wherein the belt is attachable to, and detachable from, a seat back.

8. The device of claim 1, wherein the belt provides the upward force by moving an attachment mechanism that couples the belt and at least one of a seat back surface and a lumbar support upward with respect to the seat back surface.

9. The device of claim 1, wherein the belt comprises at least one of:
a seat-back apparatus comprising a motor adapted to provide the upward force;
a seat-back apparatus comprising a pneumatic system adapted to provide the upward force;
a seat-back apparatus comprising a hydraulic system adapted to provide the upward force;
a seat-back apparatus comprising a spring system adapted to provide the upward force; and
a seat-back apparatus comprising a rack and pinion system adapted to provide the upward force.

10. The device of claim 1, wherein:
the belt includes one of a shirt, a vest and a jacket; and
the at least one sensor is a plurality of sensors, and at least one of the sensors is on the one of the shirt, the vest and the jacket.

11. The device of claim 1, wherein the at least one sensor comprises at least one of at least one pressure sensor and at least one flex sensor.

12. An ergonomic posture assisting system, comprising:
a belt adapted to encircle a torso of a user and adapted to selectively attach to, and detach from, an attachment mechanism of a seat back surface;
at least one sensor adapted to provide at least one measurement signal to a controller; and
a posture assistance system controlled by the controller and adapted to provide an upward force to a portion of the belt.

13. The ergonomic posture assisting system of claim 12, wherein the controller is adapted to signal to the user when a posture of the user is determined to be outside an acceptable range based on the at least one pressure measurement signal.

14. The ergonomic posture assisting system of claim 13, wherein the controller signals to the user when a posture of the user is determined to be outside the acceptable range using at least one of a sound and a vibration.

15. The ergonomic assisting system of claim 12, wherein the posture assistance system provides the upward force to the portion of the belt by moving the attachment mechanism upward with respect to the back surface of the seat.

16. The ergonomic assisting system of claim 12, further comprising the controller, wherein the controller communicates with the posture assistance system.

17. The ergonomic assisting system of claim 12, wherein the attachment mechanism of the seat back surface is coupled to at least one vertically oriented rail in the seat.

18. The ergonomic assisting system of claim 17, wherein the attachment mechanism is adapted to move up and down on the at least one vertically oriented rail in response to one of a motor, a pneumatic system, a hydraulic system, and a spring system.

19. The ergonomic assisting system of claim 12, wherein at least one of the attachment mechanism of the seat back surface and the belt comprise a funneling system adapted to guide the belt into engagement with the attachment mechanism.

20. The ergonomic assisting system of claim 12, wherein at least one of the attachment mechanism of the seat back surface and the belt comprise a magnet system adapted to facilitate coupling of the attachment mechanism and the belt.

21. The ergonomic assisting system of claim 12, wherein the attachment mechanism comprises at least one of an electrical connection adapted to transmit at least one of power and data and an air pressure connection.

22. The ergonomic assisting system of claim 12, wherein the attachment mechanism comprises a locking mechanism adapted to respond to at least one of a collision sensor, a speed sensor, and an accelerometer.

23. A method for assisting posture of a user, comprising:
selecting an assistance amount; and
providing an upward force to a torso of the user, an amount of the upward force being based on the selected assistance amount and varying over time.

24. The method of claim 23, further comprising determining at least one measurement signal corresponding to a pressure between the user and at least one of a seat cushion and a seat back surface when the user is prompted; and
wherein the amount of the upward force is further based on the at least one measurement signal.

25. The method of claim 24, wherein the at least one measurement signal is determined by at least one of a pressure sensor and a flex sensor.

26. The method of claim 24, wherein the at least one measurement signal is a plurality of measurement signals.

27. The method of claim 23, further comprising:
providing a feedback signal to the user based on the at least one measurement signal;
wherein the feedback signal comprises at least one of a sound and a vibration.

28. The method of claim 27, wherein:
the feedback signal is provided to alert the user that a posture of the user is determined to be outside an acceptable range; and
after the posture of the user is determined to be outside the acceptable range, further comprising determining a further at least one measurement signal corresponding to a further pressure between the user and the seat when the user sits in the seat in an adjusted posture.

29. The method of claim 28, wherein the adjusted posture of the user is identified based in part on a speed at which the users assumes the adjusted posture after the feedback signal is provided to the user.

30. The method of claim 23, wherein the assistance amount is selected by at least one of the user and a controller having a memory including data from a prior use by the user.

31. A device, comprising:
a belt adapted to encircle a torso of a user; and
an attachment mechanism adapted to removably attach the belt to a vehicle; and
a locking mechanism adapted to prevent the attachment mechanism from disconnecting in response to a signal.

32. The device of claim 31, wherein the locking mechanism is adapted to prevent the attachment mechanism from disconnecting in response to a signal from a sensor in the vehicle.

33. The device of claim 32, wherein the sensor comprises at least one of a collision sensor, an accelerometer, and a vehicle speed sensor.

34. The device of claim 33, wherein the signal from the sensor is in response to at least one of a collision force, an acceleration, a deceleration, and a vehicle speed.

35. The device of claim 31, wherein the belt comprises a size-adjustable internal belt along a bottom portion that is adapted to distribute force over the user when the belt is attached to the vehicle.

36. The device of claim 31, wherein at least a portion of the attachment mechanism is situated on a back surface of the belt.

37. The device of claim 31, wherein at least a portion of the attachment mechanism comprises a seat including a bottom surface and a back surface, the seat being attached to the vehicle.

38. The device of claim 31, wherein:
the device is adapted to aid a posture of the user; and
the device is further adapted to provide an upward force to at least part of a spinal region of the user in response to a signal from a controller.

39. The device of claim 38, further comprising at least one pressure sensor adapted to provide at least one pressure measurement signal to the controller.

40. The device of claim 31, wherein the belt comprises one of a shirt, a vest, and a jacket.

41. A posture feedback system comprising:
a lumbar support adapted to be positioned on a chair;
a sensor arranged on the lumbar support;
a controller adapted to receive a measurement signal from the sensor; and
feedback means controlled by the controller.

42. The device of claim 41, wherein the sensor comprises at least one of a flex sensor and a pressure sensor.

43. The device of claim 42, wherein the sensor comprises at least one of a plurality of flex sensors and a plurality of pressure sensors.

44. The device of claim 41, wherein the lumbar support comprises a chair back.

45. The device of claim 41, wherein a deviation in posture of a user above a preset threshold activates the feedback means.

46. The device of claim 45, wherein the feedback means is one of a sound source and a vibration source.

47. A device for monitoring a posture of a user, comprising:
a belt adapted to encircle a torso of the user;
at least one flex sensor adapted to provide at least one measurement signal to the controller; and
a feedback system activated by the controller to alert the user that a posture of the user is determined to be outside an acceptable range based on the at least one measurement signal.

48. The device of claim 47, wherein the feedback system signals to the user with at least one of a sound and a vibration.

49. The device of claim 47, wherein the posture of the user is determined to be outside the acceptable range when at least one of:
the at least one measurement signal exceeds a first predetermined threshold;
the at least one measurement signal falls below a second predetermined threshold; and
the at least one measurement signal is a plurality of measurement signals satisfying a preprogrammed model.
